(19) United States
(12) Patent Application Publication
(10) Pub. No.: US 2020/0345653 A1
(43) Pub. Date: Nov. 5, 2020

(54) Transdermal Composition

(71) Applicants: KENDA HANSEN, APTOS, CA (US); KATHLEEN GRAVE, APTOS, CA (US)

(72) Inventors: KENDA HANSEN, APTOS, CA (US); KATHLEEN GRAVE, APTOS, CA (US)

(21) Appl. No.: 16/402,128

(22) Filed: May 2, 2019

(51) Int. Cl. A61K 9/70; A61K 31/05; A61K 36/185; A61K 9/00; A61K 31/355; A61K 36/53; A61K 36/76; A61K 31/136; A61K 36/87; A61K 38/48; A61K 31/10

(52) U.S. Cl. CPC A61K 9/7023; A61K 31/05; A61K 36/185; A61K 9/0014; A61K 31/355; A61K 31/10; A61K 36/76; A61K 31/136; A61K 36/87; A61K 38/4873; A61K 36/53

(57) ABSTRACT

A formulation include a cannabinoid extract that is pharmaceutically effective via topical application. A unit dose having a cannabinoid component in the range of from 1 milligram to 100 milligram is included within an oil or other solution sourced from the same plant or same type of plant from which the cannabinoid component is sourced, another cannabinoid-producing plant, and/or one or more suitable oils known in the art. One version contains the cannabinoid extract provided in a unit dose within the range by volume of a pharmaceutically-active composition of from less than 0.3 milliliter to 3.0 milliliter that contains a cannabinoid component preferably in the range of from 1 milligram to 100 milligram. The unit dose may include Jojoba oil, dimethyl sulfoxide, bromelain, olive oil, fulvic acid, and/or Vitis vinifera seed oil.

### FIELD OF THE INVENTION

This disclosure relates generally to compositions that include at least one cannabinoid, and particularly to formulated cannabinoid mixtures that enable transdermal delivery of topically applied volume of at least one cannabinoid.

### BACKGROUND OF THE INVENTION

The medicinal use of oils and extracts derived from cannabis plant material has been growing in popularity. For example, pharmacologically-active compounds in cannabis plant material including, but not limited to, delta-9-tetrahydrocannabinol (or THC) and cannabidiol (CBD) have been shown to reduce the effects of nausea and vomiting caused by certain chemotherapy treatments.

Research has also shown the ability of cannabinoids and other compounds found in cannabis to stimulate bone growth, relieve pain, aid sleep, inhibit bacterial cell growth, inhibit cancer cell growth, and alleviate or otherwise reduce the symptoms of cancer, epilepsy, autoimmune disease, neurodegeneration, Alzheimer's disease, Lyme disease, post-traumatic stress disorder, and inflammation. Furthermore, extracts of cannabis plant material, whether ingested or inhaled, have also been shown to have therapeutic effects in patients with glaucoma, dysmenorrhea, migraines, anxiety disorders, or a combination thereof.

However, cannabis oil is often highly viscous, making it difficult to work with and load into new delivery devices such as vaporizers and E-cigarettes. In addition, such oils, when vaporized or smoked, are often rough on a patient's throat and may induce coughing or gagging.

Furthermore, cannabinoids degrade rapidly in the body, reducing the efficacy of cannabinoids. Ways of improving efficacy, bioavailability, and bioactivity of cannabinoids are desired.

There is therefore a long-felt need for compositions containing a cannabinoid volume that are effective, stable and easily delivered into an intended recipient's body. It is an object of the present invention to provide topically-applicable compositions of matter that offer the ease of use of transdermal delivery of a cannabinoid ingredient.

### SUMMARY OF THE INVENTION

Towards this object and other objects that are made obvious to one of ordinary skill in the art in light of the present disclosure, the present invention may be or be comprised within any of a number of formulations that include a cannabinoid extract and are pharmaceutically effective via topical application. One or more alternate preferred embodiments of the present invention may optionally provide a unit dose having a cannabinoid component in the range of from 1 milligram to 100 milligram included within an oil or other solution sourced from the same plant from which the cannabinoid component is sourced, another cannabinoid-producing plant, and/or one or more suitable oils known in the art.

A first alternate preferred embodiment of the present invention contains the cannabinoid extract provided in a unit dose within the range by volume of a pharmaceutically active composition of from less than 0.3 milliliter to 3.0 milliliter that contains a cannabinoid component preferably in the range of from 1 milligram to 100 milligram.

Various other alternate preferred embodiments of the present invention may include one or more additional ingredients, within the cannabinoid extract and/or added in addition to the cannabinoid extract, such as a volume of Simmondsia chinensis (Jojoba) oil, a volume of Vitis vinifera seed oil, a volume of fulvic acid liquid concentrate, a volume of Capric triglycerides, a volume of dimethyl sulfoxide (hereinafter, “DMSO”) and/or a volume of bromelain. Simmondsia chinensis (Jojoba) oil may be included as an alternative to Vitis vinifera (grape) seed oil in other additional alternate preferred embodiments of the present invention.

Various still alternate preferred embodiments include one or more alternate or additional ingredients, to include (a.) a volume of industrial, cosmetic or pharmaceutical-grade dimethyl sulfoxide; (b.) a volume of Arnica montana powder or extract; (c.) a volume of Vaccinium corymbosum seed oil; (d.) a volume of Vitamin E; (e.) a volume of Lavandula angustifolia oil; (f.) a volume of Mentha piperita oil; and/or (g.) a volume of Populus balsamifera; (h.) a volume of lidocaine; and/or (i.) a volume of Hamamelis virginiana.

Still other alternate preferred embodiments of the present invention may include one or more alternate or additional ingredients, to include an additional suitable permeation enhancer known in the art, to include Simmondsia chinensis (Jojoba) oil, Vitis vinifera (grape) seed oil, bromelain, fulvic acid, and DMSO.

It is understood that additional alternate preferred embodiments of the present invention may include one or more alternate or additional ingredients that are suitable for and/or legally permitted for introduction into the biological systems of animals and other non-human beings, to include DMSO.

Still other alternate preferred embodiments of the present invention may include one or more alternate or additional ingredients, to include pharmaceutically-acceptable excipients to create a transdermal dosage form selected from the group consisting of: gels, ointments, cataplasms, poultices, pastes, creams, lotions, plasters, and jellies.

Even other alternate preferred embodiments of the present invention may comprise inclusion of a liposomal delivery mixture in a unit dose formulation, such as hydrogenated soybean phosphatidylcholine or other suitable liposomal delivery mixtures, to include, or optionally, additionally or alternatively consist of, (a.) Methoxy Poly(ethylene glycol)-block-poly-L-lactide, (b.) Methoxy Poly(ethylene glycol)-block-poly(ε-caprolactone), (c.) Maleimide-(CH2)2CONH(CH2)3O-PEG-(CH2)3-NHCO—(CH2)2-Maleimide, Mw 10,000, (d.) DSPE:1,2-Distearoyl-sn-glycero-3-phosphoethanolamine, (e.) DSPC:1,2-Distearoyl-sn-glycero-3-phosphocholin, and/or (f.) Polysorbate 80, and/or any suitable nanoliposomal or liposomal delivery substance, known in the art. It is understood that the scope of meaning of the term “nanoliposomal delivery mixture” as applicable to and applied within this disclosure includes a single substance or a combination of substances.

Even other additional alternate preferred embodiments of the present invention are formulated for topical, sublingual and/or ingested application a non-human subject and may optionally include DMSO of industrial, cosmetically or pharmaceutical grade.

Yet other alternate preferred embodiments of the present invention may include an occlusive body (hereinafter, “the invented patch”) configured for delivering cannabidiol into systemic circulation of a wearer of the invented patch. The invented patch may include (a) an impermeable backing; (b) a rate-controlling microporous membrane, said backing and membrane defining a cavity there between; (c) the cannabidiol disposed within the cavity; (d) a volume of permeation enhancer disposed within the cavity, the volume of permeation enhancer selected from the permeation enhancer group consisting of Simmondsia chinensis oil, DMSO″, Vitis vinifera seed oil, bromelain, and fulvic acid; and/or (e) a viscous, flowable gel confined between the backing and the membrane within the cavity for immobilizing the cannabinoid and the volume of permeation enhancer. The invented patch may optionally further include an adhesive for attaching the invented patch to a patient's skin, whereby the cannabinoid and permeation enhancer are released through the membrane to the patient's skin. The optional membrane of the invented patch may present an exterior surface coated with an adhesive.

The invented method optionally includes the aspect of applying ultra-sonification to emulsify and homogenize one or more oils and one or more liposomes to form a nanoliposome blend. Optionally one or more oils may first be blended and/or emulsified, after which one or more volumes of liposomes are added to the emulsified oils and the resultant formulation is again emulsified by ultra-sonification. The cannabinoid may be added to the oils and/or an oil and liposome formulation before or after either ultra-sonification application.

In yet additional alternate preferred embodiments of the invented method, materials added to form a resultant substance are preferably maintained within a temperature range between 35 degrees Fahrenheit and 90 degrees Fahrenheit. In yet other additional alternate preferred embodiments of the invented method, vitamin E and grape seed oil are first blended at 80 degrees Fahrenheit and added to one or more oils.

In even other alternate preferred embodiments of the invented method, the cannabinoid extract may be extracted from flowers, leaves, and stems of cannabis plants.

In even other additional alternate preferred embodiments of the invented method, the cannabinoid may be extracted by Ultra-sonification extraction, spagyric extraction, and/or CO₂ extraction. Ultra-sonification extraction refers to the use of ultrasonic waves. It is understood that may also be used in the invented method to emulsify and homogenize oils, cannabinoids and/or liposomes. It is understood that ultrasonic extractors that accomplish do not rely on heat and may be applied without exceeding 60 degrees Celsius of a subject material. Ultra-sonification extraction is applied in yet additional other alternate preferred embodiments of the invented method to emulsify and homogenize a formulation-in-process, resulting in a nanoliposome blend. Oils may be emulsified and homogenized first, and then liposomes may then be added and emulsified and homogenized with the emulsified and homogenized oils.

Spagyric extraction uses grape ethanol to extract tinctures of terpenes, cannabinoids, and phytochemicals from hemp.

The CO₂ extraction method is a gentle, low temperature, alcohol-free extraction process and is free of harmful solvents.

The terms “mixture”, “combination”, “formulation”, and “blend” are each defined herein to have a range of meaning that includes a substance made of two or more different materials, such as solutions, emulsifications, stirred materials, mixed materials, combinations, chemically-reacted or -reacting materials, compounded materials, adhering materials, and/or other suitable substances comprising chemical substances located within a gel, ointment, cataplasm, poultice, paste, cream, lotion, plaster, or jelly. The terms “mixing” “combining” and “blending” are each defined herein to have a range of meaning that includes a process of one or more steps that joins or co-locates two or more differing materials to form one or more suitable substances type known in the art, such as solutions, emulsifications, blends, stirred materials, mixed materials, combinations, chemically-reacted or -reacting materials, compounded materials, adhering materials, and/or other suitable substances comprising chemical substances located or positioned within a gel, ointment, cataplasm, poultice, paste, cream, lotion, plaster, or jelly.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### INCORPORATION BY REFERENCE

All publications, patents, and patent applications mentioned in this specification are herein incorporated by reference to the same extent as if each individual publication, patent, or patent application was specifically and individually indicated to be incorporated by reference. U.S. Pat. No. 10,206,888 titled “CANNABIS-BASED THERAPEUTIC PRODUCT FOR TREATMENT OF CHRONIC PAIN” issued on Feb. 19, 2019 to Inventors Vu, et al.; U.S. Pat. No. 10,143,755 titled “Anhydrous hydrogel composition and delivery system” issued on Dec. 4, 2018 to Inventors Borja, et al.; U.S. Pat. No. 10,188,628 titled “RELEASE COMPOSITION FOR DERIVATIVES OF CANNABACEAE” issued on Jan. 29, 2019 to Inventors Kershman et al; U.S. Pat. No. 9,827,322 titled “Medication dispensing system” issued on Nov. 28, 2017 to Inventor Naheed, Shabana; U.S. Pat. No. 9,827,282 titled “Medication dispensing system” issued on Nov. 28, 2017 to Inventor Naheed, Shabana; U.S. Pat. No. 9,029,423 titled “PHARMACEUTICAL FORMULATION” issued on May 12, 2015 to Inventor Whittle, Brian Anthony; U.S. Pat. No. 8,435,556 titled “Transdermal delivery of cannabidiol” issued on May 7, 2013 to Inventors Stinchcomb et al.; U.S. Pat. No. 6,328,992 titled “Cannabinoid patch and method for cannabis transdermal delivery” issued on Dec. 11, 2001 to Inventors Brooke, et al.; U.S. Pat. No. 6,113,940 titled “Cannabinoid patch and method for cannabis transdermal delivery” issued on Sep. 5, 2000, to Inventors Brooke, et al.; U.S. Pat. No. 5,716,928 titled “Use of essential oils to increase bioavailability of oral pharmaceutical compounds” issued on 1998 Feb. 10, to Inventors Benet, et al.; U.S. Pat. No. 5,254,346 titled “Occlusive body for administering a physiologically active substance” issued on Oct. 19, 1993 to Inventors Tucker, et al.; and U.S. Pat. No. 5,064,654 titled “Mixed solvent mutually enhanced transdermal therapeutic system” issued on Nov. 12, 1991 to Inventors Berner et al. are incorporated herein by reference in their entirety and for all purposes.

### BRIEF DESCRIPTIONS OF THE DRAWINGS

The invention will now be further and more particularly described, by way of example only, and with reference to the accompanying drawings in which:

FIG. 1 shows a first preferred embodiment of the present invention of exemplary transdermal patch comprising the present invention;

FIG. 2 is process chart presenting aspects of preparing alternate preferred embodiments of the invented oil mixture and presenting additional optional and alternative ingredients and process steps of FIG. 1;

FIG. 3A is a process chart presenting aspects of preparing a first preferred embodiment of the invented oil mixture of FIG. 1 containing at least a volume cannabinoid and a volume of Simmondsia chinensis (Jojoba) oil;

FIG. 3B is a process chart presenting aspects of preparing a second preferred embodiment of the invented oil mixture of FIG. 1 containing at least a volume of cannabinoid and a volume of Vitis vinifera seed oil;

FIG. 3C is a process chart presenting aspects of preparing a third preferred embodiment of the invented oil mixture of FIG. 1 containing at least a volume cannabinoid and a volume of liquid fulvic acid;

FIG. 3D is a process chart presenting aspects of preparing a fourth preferred embodiment of the invented oil mixture of FIG. 1 containing at least a volume cannabinoid and a volume of bromelain;

FIG. 3E is a process chart presenting aspects of preparing a fifth preferred embodiment of the invented oil mixture of FIG. 1 formulated for sublingual or ingested consumption or ingestion by a subject;

FIG. 4 is an additional process chart of the presenting aspects of preparing alternate preferred embodiments of the invented oil mixture and presenting additional optional and alternative ingredients and process steps of FIG. 1;

FIG. 5 is an additional process chart of the presenting aspects of preparing alternate preferred embodiments of the invented oil mixture of FIG. 1 and presenting additional optional process steps of adding the invented oil mixture to a lotion, cream, or gel;

FIG. 6 is a table of unit dose formulation listings and presenting both an exemplary unit dose formulations of alternate preferred embodiments of the invented oil mixture of FIG. 2 in one column and a range of ingredient magnitudes thereof in another column; and

FIG. 7 is a formulary table 700 of unit dose formulation listings and presenting both an exemplary unit dose formulation of alternate preferred embodiments of the invented ingestible mixture of FIG. 3E.

### DETAILED DESCRIPTION

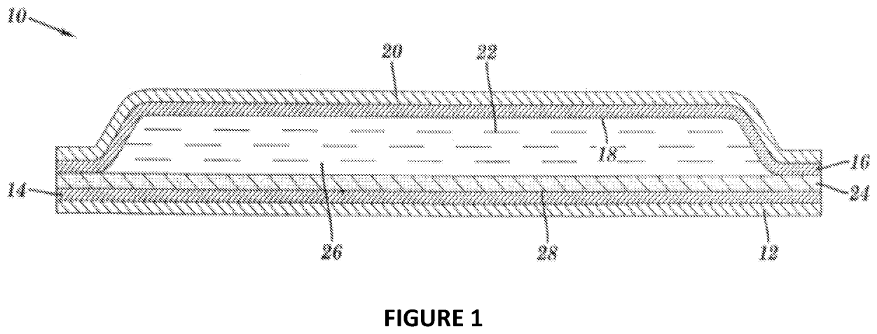

Referring now generally to the Figures and particularly to FIG. 1, FIG. 1 presents an occlusive body 10 of the present invention. Occlusive body 10 includes impermeable backing 16 having optional polyester face 18, cannabinoid composition 22 (hereinafter, “invented oil mixture” 22), and rate-controlling microporous membrane 24. Theate-controlling microporous membrane 24 is shown heat-sealed around the periphery of its upper face to optional polyester face 18 of impermeable backing 16. However, other methods of sealing rate-controlling microporous membrane 24 to impermeable backing 16 (or to optional polyester face 18 of impermeable backing 16) can be employed. Impermeable backing 16 is illustrated as including optional aluminized layer 20 on the outer face thereof. Impermeable backing 16 and rate-controlling microporous membrane 24 define cavity 26, and the invented oil mixture 22 is disposed in cavity 26. Over time, the cannabinoid contained in the invented oil mixture 22 permeates through rate-controlling microporous membrane 24 and optional adhesive layer 14, which is illustrated as being coated on the lower face of rate-controlling microporous membrane 24.

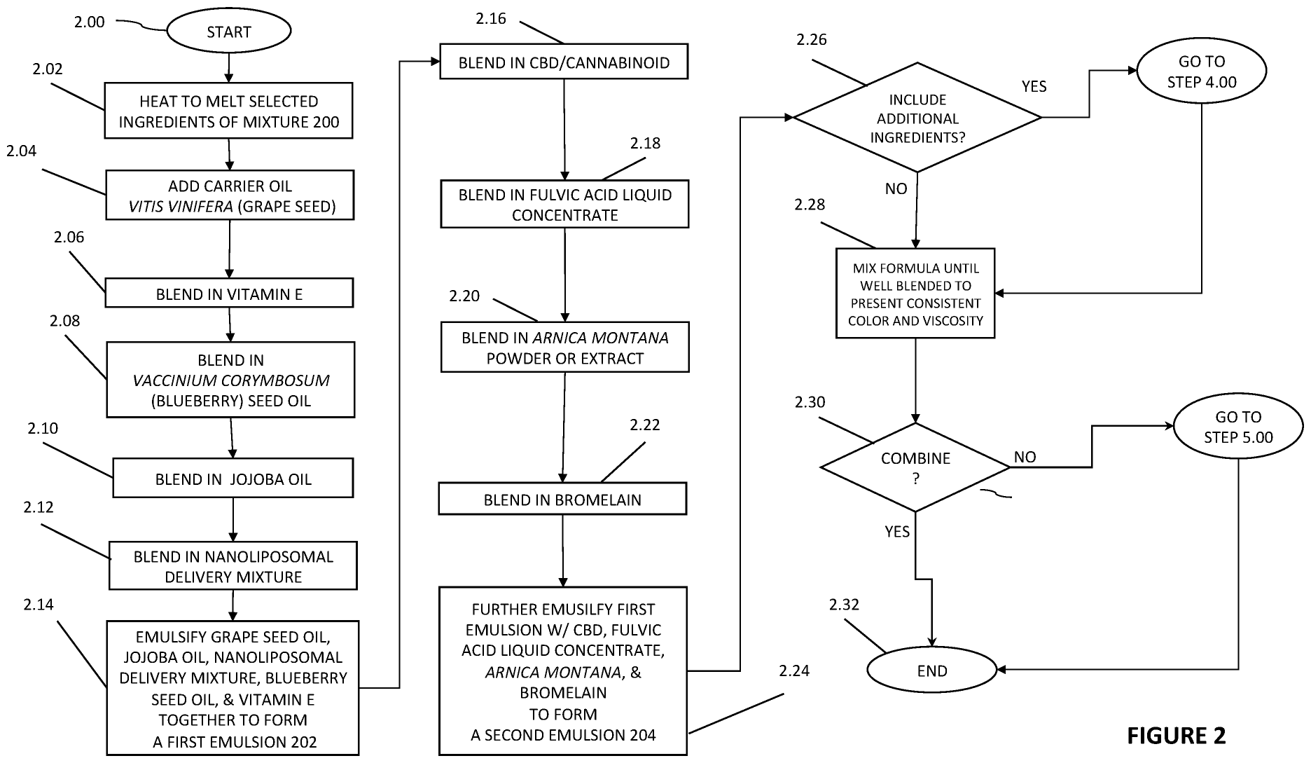

As indicated above, the invented oil mixture 22 contains at least one cannabinoid. Cannabinoids for use in the occlusive body of the present invention can be selected from the group consisting of .DELTA..sup.9-THC, .DELTA..sup.8-THC, cannabinol, cannabidiol, nabilone, levonantradol, (−)-HU-210, (+)-HU-210, 11-hydroxy-.DELTA..sup.9-THC, .DELTA..sup.8-THC-11-oic acid, CP 55,940, and R(+)-WIN 55,212-2. Preferably, cannabinoids for use in the occlusive body of the present invention are selected from the group consisting of cannabinol, cannabidiol, nabilone, levonantradol, (−)-HU-210, (+)-HU-210, 11-hydroxy-.DELTA..sup.9-THC, .DELTA..sup.8-THC-11-oic acid, CP 55,940, and R(+)-WIN 55,212-2. Mixtures of these and other suitable cannabinoids known in the art can also be employed. Preferably, the cannabinoid is selected such that it is sufficiently hydrophobic to pass through rate-controlling microporous membrane 24. In addition, the invented oil mixture 22 can also include other inert or active materials, such as those discussed above with regard to topical formulations disclosed in FIG. 2 through FIG. 6 and/or such as those described in relation to the occlusive body 10.

The invented oil mixture 22 may include an aqueous medium, which can contain a water- and oil-miscible solvent. The cannabinoid composition can also contain a material which enhances the cannabinoid's permeation of the skin. Depending on the nature of the chosen solvent, the solvent can also act as the permeation enhancer, or a separate permeation enhancer having the desired miscibility can be added to the cannabinoid composition. Illustrative permeation enhancers that can be used in the occlusive bodies of the present invention include ethanol and oleic acid. Preferably the cannabinoid is present together with at least one diluent so that the cannabinoid accounts for no more than about 25% by weight of the contents of the occlusive body's cavity.

The cannabinoid composition can also include one or more inhibitors of cannabinoid metabolism, particularly in cases where inhibition of cutaneous metabolism is needed to increase therapeutic drug levels. Such inhibitors of cannabinoid metabolism can include inhibitors of the P450 enzymes or other identified critical enzymatic processes. Suitable inhibitors of cannabinoid metabolism include, for example, essential oils which inhibit the activity of cytochrome P450 3A in the skin, such as those described in U.S. Pat. No. 5,716,928 to Benet et al., which is hereby incorporated by reference. Some of these essential oils may also act as transdermal penetration enhancers, thus providing a dual mechanism of percutaneous penetration increase.

Rate-controlling microporous membrane 24 can optionally be made of a single-ply material, or it can be made of a multi-ply material. Only the inner layer of such a membrane needs to be hydrophobic (in the case that the cavity contents are hydrophilic) or hydrophilic (in the case that the cavity contents are hydrophobic). Thus, in one embodiment, an additional permeable membrane is in contact with an exterior surface 28 of rate-controlling microporous membrane 24, and the additional permeable membrane has wetting properties which are the same as, or different from, the wetting properties of rate-controlling microporous membrane 24.

It is believed that the greater the difference in wetting properties between the cavity contents and rate-controlling microporous membrane 24 (or the innermost layer of rate-controlling microporous membrane 24 if a multi-ply membrane is used), the wider the range of useful solvents and the more linear the release of the drug. Accordingly, it is desirable to employ either a strongly hydrophobic or a strongly hydrophilic rate-controlling microporous membrane 24 (or the innermost layer of the rate-controlling microporous membrane 24 if a multi-ply membrane is used) in conjunction with strongly hydrophilic cavity contents and strongly hydrophobic cavity contents, respectively. It should be noted that the cavity contents can be made hydrophilic by adding surface-active agent, such as an anionic surface-active agent (e.g., sodium lauryl sulphonate), a cationic surface-active agent (e.g., cetrimide), or a non-ionic surface-active agent (e.g., TWEEN 20™.

Occlusive body 10 can also have an outer layer of an impervious material, such as a layer of aluminum foil or other metal or plastic laminate, to prevent seepage or leaching of the contents of the cavity 26. The cavity side of rate-controlling microporous membrane 24 can be faced with an area-reducing mesh formed, for example, from a non-woven fabric or from a perforated impermeable material such as aluminum foil.

Rate-controlling microporous membrane 24 can be made of any suitable membrane material, such as a hydrophobic and microporous membrane material, for example, CELGARD™ microporous membrane material 2500 polypropylene of thickness 0.025 mm (1 mil) and pore size 0.4-0.04 microns.

The exterior surface 28 of the rate-controlling microporous membrane 24 (i.e., the face distant from cavity 26) can optionally be coated with adhesive layer 14, for example, having a thickness of, for example, about 30 micrometers. Any suitable dermatologically-acceptable, pressure-sensitive adhesive that does not react chemically with the cavity contents or prevent passage of the cannabinoid through the membrane from being rate-controlling can be used for adhesive layer 14. Thus, the adhesive can be chosen such that the cannabinoid passes reasonably rapidly through adhesive layer 14, though some retardation is acceptable in practice. The adhesive can be, for example, an elastomeric silicone polymer. Other suitable adhesives include polyisobutylenes and acrylates. Optional release liner 12, such as a sheet of release coated paper or other material, can be used to cover adhesive layer 14 until the occlusive body 10 is to be used, thus preventing cannabinoid permeation prior to contacting occlusive body 10 with the subject's skin. Immediately prior to use, release liner 12 is stripped from adhesive layer 14, and occlusive body 10 is adhered to the subject's skin (e.g., of the arm) (not shown) by the exposed adhesive layer 14.

It is to be understood that adhesive layer 14 is but one of many suitable ways for attaching the occlusive body 10 to the subject's skin. For example, as an alternative to using adhesive layer 14, a separate tape or bandage material can be employed to attach the occlusive body of the present invention to the subject's skin.

The occlusive body of the present invention can further include a viscous flowable gel which is disposed within the occlusive body's cavity and which immobilizes the cannabinoid within the cavity. Such gel formulations can be useful to reduce the likelihood of abrupt absorption of the cannabinoid in the event of sudden rupture of the cavity and release of the cavity contents onto the skin. Suitable gel formulations can be achieved by making the viscosity of the cavity contents sufficiently high such that they are resistant to spreading in the event of cavity puncture. Illustratively, methyl cellulose in water can be used as a viscosity modifier in such gel formulations. In certain situations, the use of methyl cellulose in combination with the cannabinoid composition can also be advantageous in that the methyl cellulose can also function as a surface-active agent to enhance the hydrophilicity of the cavity contents.

In this invention, the rate-controlling microporous membrane can be a hydrophobic microporous material, such as hydrophobic microporous polypropylene or polyethylene. The cavity contents can illustratively include, in addition to the cannabinoid, a wetting agent water based gel formed, for example, using methyl cellulose. As a further illustration of an occlusive body of the present invention, the rate-controlling microporous membrane can be a hydrophobic microporous polypropylene membrane and the cavity can contain, in addition to the cannabinoid, a water-based gel containing an amount of methyl cellulose (e.g., 5%) which gives a linear release of the cannabinoid while retaining water and solids. It may be expedient to dissolve the cannabinoid in an appropriate pharmaceutically-acceptable vehicle, which will carry the active substance through the rate-controlling microporous membrane. Moreover, the rate of delivery of the cannabinoid through the rate-controlling microporous membrane into the blood stream of the subject can be varied by varying the surface area, thickness, and composition of the membrane; by varying the weight ratio of cannabinoid-to-vehicle; and by varying the hydrophilicity of the cavity contents.

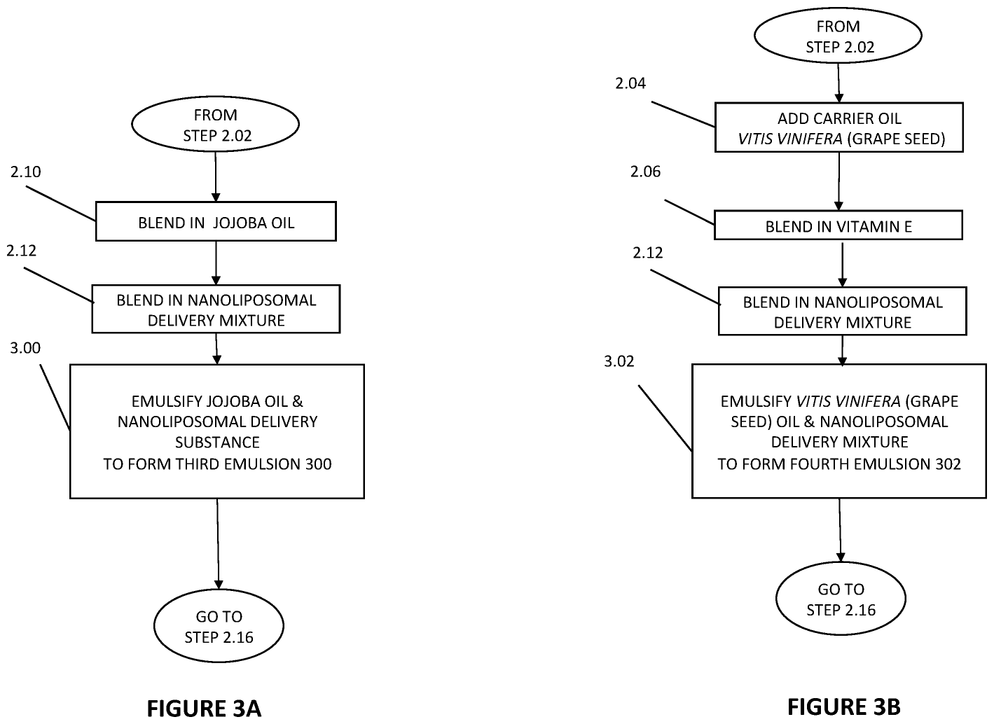

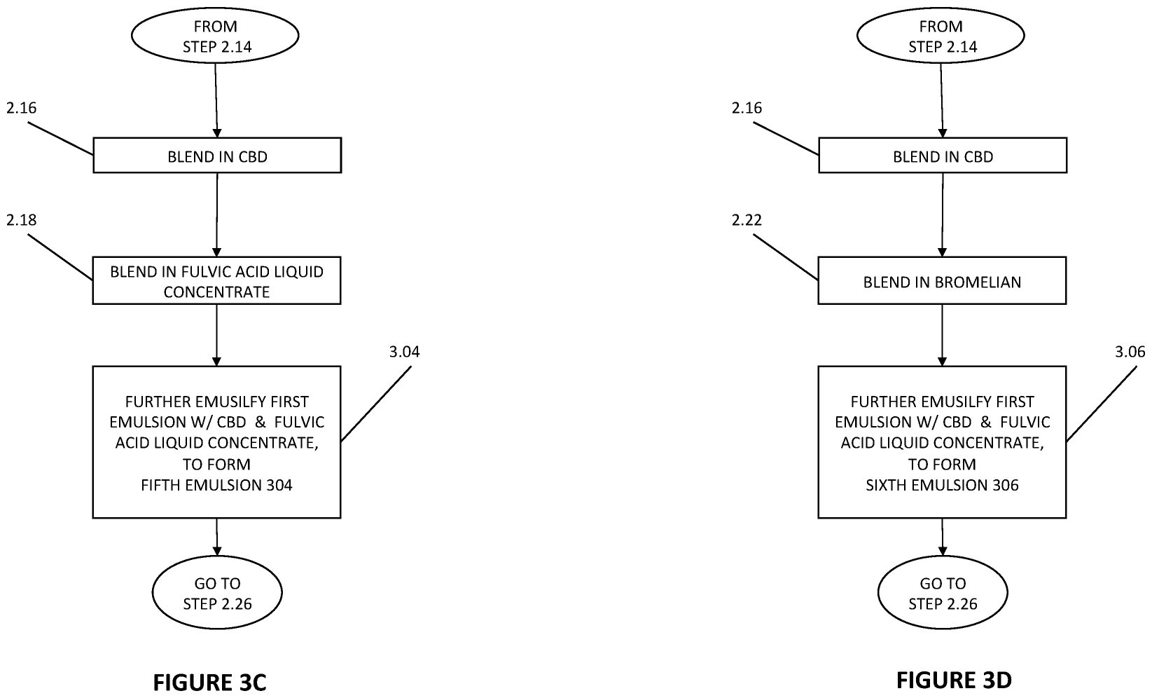

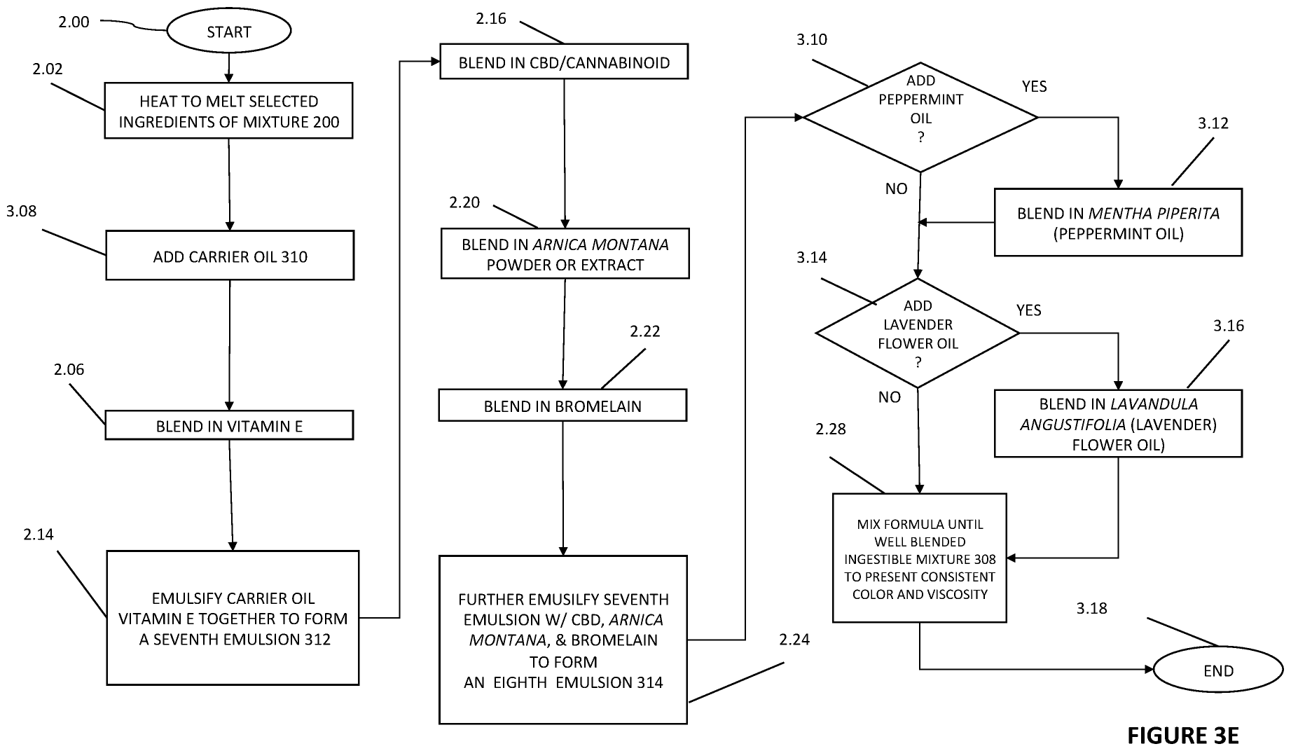

Referring now generally to the Figures, and particularly to FIG. 3, FIG. 3 is a process chart displaying a preferred process for preparing several alternative variations of the invented oil mixture 22. It is understood that the term “compounder” will be used in the descriptions of the methods of FIGS. 2, 3A, 3B, 3C 3D, 3E, 4 and 5, wherein the range of meaning of “compounder” (not shown) includes a single human preparer, a group of humans, one or more automated processes in singularity, combination and/or on concert or collaboration are preparing one of a wide variety of alternate preferred embodiments of the invented mixture 22.

The method of FIGS. 2, 3A, 3B, 3C 3D, 3E, 4 and 5 consists overall of the disclosed ingredients alternatively or optionally being added into a same single mixture in a sequence. This single mixture being changed or added to with each step, and comprising in each instance all the ingredients and changes cumulatively added in prior steps, is henceforth referred to throughout as “the present mixture”. It is understood that the term “the present mixture” 200 describes the partial plurality of ingredients of the invented oil mixture 22 that are being combined, mixed, and/or emulsified in the method of FIGS. 2, 3A, 3B, 3C 3D, 3E, 4 and 5, with the eventual resultant mixture of formulating a preferred embodiment of the invented oil mixture 22.

In a first step 2.00 of the method of FIG. 2, a process of formulating a present mixture is initiated. In step 2.02, the compounder selectively heats one or more of the ingredients disclosed herein to a preferred temperature for the purposes of the effectiveness of the process steps of the method of FIGS. 2, 3A, 3B, 3C, 3D, 3E, 4, and 5. It is understood that the ingredients selected for use in the method of FIGS. 2, 3A, 3B, 3C, 3D, 3E, 4, and 5 are preferably maintained within a temperature range between 35 degrees Fahrenheit and 90 degrees Fahrenheit.

In optional step 2.04 and optional step 2.06 the compounder provides and blends together the ingredients of a volume of Vitis vinifera (grape) seed carrier oil and a volume of vitamin E. It is understood that the Vitis vinifera (grape) seed carrier oil and the volume of vitamin E are preferably heated in step 2.02 to a temperature of 80 degrees F.+/−3 degrees F. In optional step 2.08 the compounder provides and blends a volume of ingredient Vaccinium corymbosum (blueberry) seed oil into the present mixture 200. In optional step 2.10 the compounder provides and blends a volume of Simmondsia chinensis (Jojoba) oil into the present mixture 200. And in optional step 2.12 the compounder provides and blends a volume of hydrogenated soybean phosphatidylcholine and/or other suitable nanoliposomal delivery mixture known in the art into the present mixture 200, to include, or optionally, additionally or alternatively consist of, (a.) Methoxy Poly(ethylene glycol)-block-poly-L-lactide, (b.) Methoxy Poly(ethylene glycol)-block-poly(ε-caprolactone), (c.) Maleimide-(CH2)2CONH(CH2)3O-PEG-(CH2)3-NHCO—(CH2)2-Maleimide, Mw 10,000, (d.) DSPE:1,2-Distearoyl-sn-glycero-3-phosphoethanolamine, (e.) DSPC:1,2-Distearoyl-sn-glycero-3-phosphocholin, and/or (f.) Polysorbate 80, and/or any suitable nanoliposomal or liposomal delivery substance, known in the art.

In step 2.14 the compounder emulsifies the present mixture 200 to create a first emulsion 202 from the collection of the ingredients contained in the present mixture 200 as of step 2.14. The compounder may optionally use a prior art ultrasonic processor in step 2.14 to form the first emulsion 202, such as a UIP 1500hdt™ (20 kHz, 15000 W) ultrasonics processor as marketed by Hielscher Ultrasonics, GMBH of Teltow, Germany, or other suitable ultrasonics processor known in the art.

In step 2.16 the compounder further blends a volume of a cannabinoid extract into the first emulsion 202. Preferably the volume of cannabinoid extract provided in step 2.16 includes one milligram (hereinafter, “mG”) to 100 mG of cannabinoid and a volume of oil in the range of from less than 0.3 milliliter (hereinafter, “mL”) to 3.0 mL of oil+/−10%.

In step 2.16 the compounder further blends a volume of a cannabinoid extract into the first emulsion 202. Preferably the volume of cannabinoid extract provided in step 2.16 includes one milligram (hereinafter, “mG”) to 100 mG of cannabinoid and a volume of oil in the range of from 10 milliliter (hereinafter, “mL”) to 30 mL of oil+/−10%.

In step 2.18 the compounder optionally further blends a volume of fulvic acid liquid concentrate into the first emulsion 202. In step 2.20 the compounder further blends a volume of Arnica montana powder or extract into the first emulsion 202. In step 2.22 the compounder further blends a volume of bromelain into the first emulsion 202.

In step 2.24 the compounder further emulsifies the first emulsion 202 to create a second emulsion 204 from the collection of the ingredients contained and added to the first emulsion 202 as of step 2.24. In the second emulsification 204 of step 2.24, the compounder may optionally use the prior art ultrasonic processor of step 2.14, such as the UIP 1500hdt™ (20 kHz, 15000 W) ultrasonics processor as marketed by Hielscher Ultrasonics, GMBH of Teltow, Germany, or other suitable ultrasonics processor known in the art, to form the second emulsion 204.

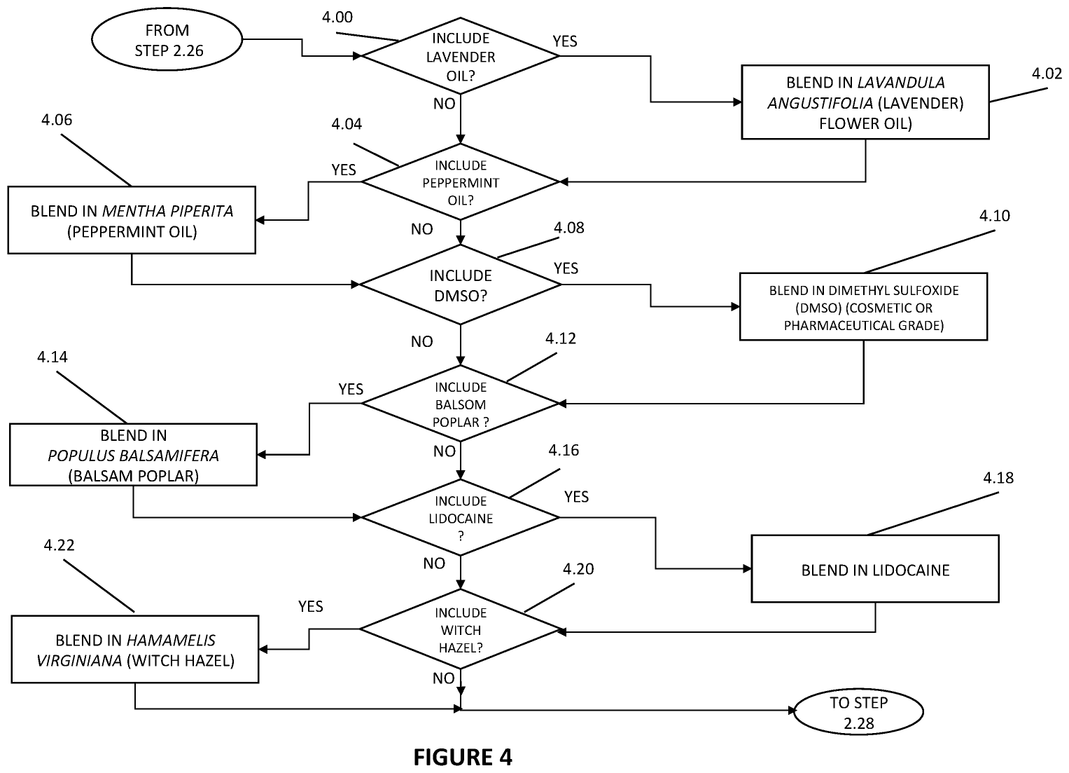

In step 2.26 the compounder elects whether or not to include one or more optional ingredients according to the desired variant of the invented oil mixture 22, and proceeds on to step 4.00 of FIG. 4 when deciding to additionally include one or more optional ingredients to the second emulsion 204. In the alternative, the compounder proceeds from step 2.26 to step 2.28 when deciding to not include one or more additional optional ingredients to the second emulsion 204, and thereupon proceeds form step 2.26 to step 2.28.

In optional step 2.28 the compounder applies a prior art mixer, such as a DeBEE 3000 ™ homogenizer mixer marketed by Bee International Corporation of South Easton, Mass., or other suitable mixer or blender known in the art, in an attempt to mix the second emulsion 204 to present a color and viscosity of the second emulsion 204, with or without one or more additional agreements as optionally blended into the second emulsion 204 in steps 4.02, 4.06, 4.10, 4.14, 4.18 and/or 4.22 of the method of FIG. 4.

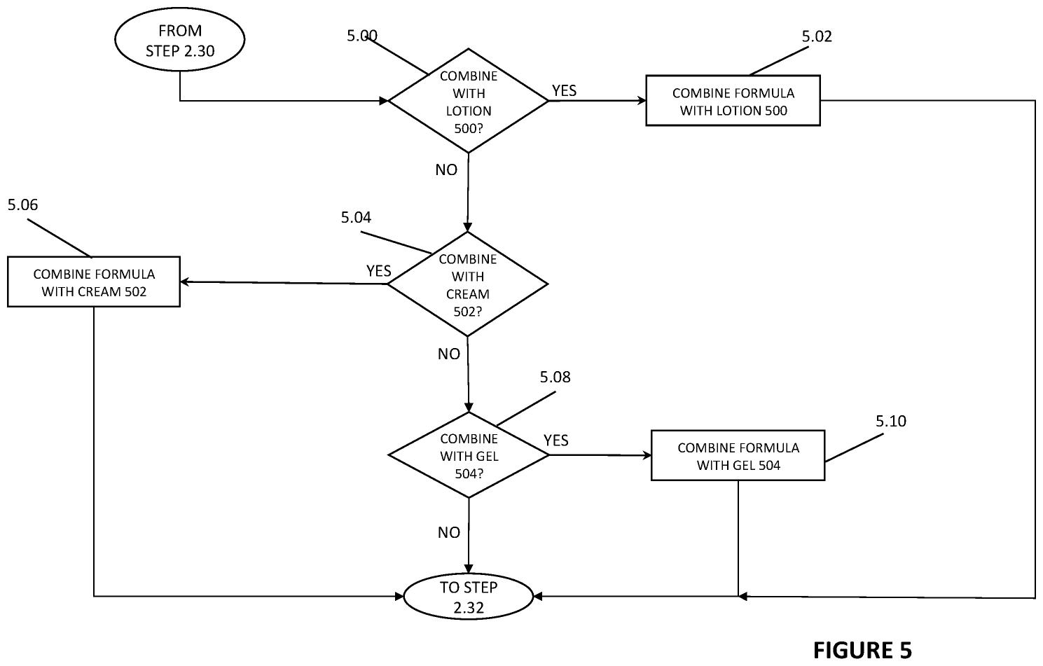

The compounder proceeds from step 2.24, optional step 2.26 or optional step 2.28 to step 2.30. In optional step 2.30, compounder elects whether to mix the second emulsion 204 in its composition as found in step 2.24 or optional step 2.26 into a topical application medium such as a lotion 500, cream 502, or gel 504 of the method steps of FIG. 5. More particularly, in step 2.30 the compounder elects whether to proceed onto step 5.00 of FIG. 5 and to mix the second emulsion 204 in its composition as found in step 2.24 or optional step 2.26 into a topical application medium. In the alternative, the compounder proceeds from step 2.30 to step 2.32 when deciding in step 2.30 to not mix the second emulsion 204 in its composition as found in step 2.24 or optional step 2.26 into a topical application medium 500, 502 & 504.

The compounder proceeds from step 2.24, optional step 2.26, optional step 2.28, optional step 2.30, or optional step 5.10 of the method of FIG. 5 to step 2.32, whereupon the formulation of the invented oil mixture 22 is designated as completed in one of a wide variety of alternate preferred embodiments of the invented oil mixture 22.

Referring now generally to the Figures and particularly to FIG. 3A, FIG. 3A is a process chart presenting aspects of preparing a first alternate preferred embodiment of the invented oil mixture 22 (hereinafter, “the third emulsion” 300). The volume of nanoliposomal delivery mixture and the volume of Simmondsia chinensis (Jojoba) oil are emulsified together in step 3.00 to form the third emulsion 300. In performing the process of FIG. 3A, the compounder proceeds from executing step 3.00 to performing step 2.16 of the method of FIG. 2.

Referring now generally to the Figures and particularly to FIG. 3B, FIG. 3B is a process chart presenting aspects of preparing fourth emulsion 302 of the invented oil mixture 22. A volume of Vitis vinifera seed oil and a volume of vitamin E are gathered in step 2.00 and each brought to a desired temperature for blending in step 2.02, e.g. 80 degrees Fahrenheit. The volume of Vitis vinifera seed oil and the volume of vitamin E are blended together in a vessel (not shown) in step 2.12. The process of FIG. 3B proceeds from step 2.12 to step 3.02 of the method of FIG. 3B to form the fourth emulsion 302. In performing the process of FIG. 3B, the compounder proceeds from executing step 3.02 to performing step 2.16 of the method of FIG. 2.

Referring now generally to the Figures and particularly to FIG. 3C, FIG. 3C is a process chart presenting aspects of preparing a fifth emulsion 304 of the invented oil mixture 22. A volume of a suitable cannabinoid material known in the art and a volume of fulvic acid liquid concentrate are blended together and optionally with the first emulsion 200, the third emulsion 300, or the fourth emulsion 302 in step 2.16 and 2.18 respectively. The volume of suitable cannabinoid material known in the art and the volume of fulvic acid liquid concentrate are thereafter emulsified together in step 3.04 optionally with the first emulsion 200, the third emulsion 300, or the fourth emulsion 302 to form the fifth emulsion 304. In performing the process of FIG. 3C, the compounder proceeds from executing step 3.04 to perform step 2.26 of the method of FIG. 2.

Referring now generally to the Figures and particularly to FIG. 3D, FIG. 3D is a process chart presenting aspects of preparing a sixth emulsion 306 of the invented oil mixture 22. A volume of a suitable cannabinoid material known in the art and a volume of bromelain are blended together and optionally with the first emulsion 200, the third emulsion 300, or the fourth emulsion 302 in step 2.16 and 2.22 respectively. The volume of suitable cannabinoid material known in the art and the volume of bromelain are thereafter emulsified together in step 3.06 optionally with the first emulsion 200, the third emulsion 300, or the fourth emulsion 302 to form the sixth emulsion 306. In performing the process of FIG. 3C, the compounder proceeds from executing step 3.06 to perform step 2.26 of the method of FIG. 2.

It is understood that the first emulsion 202 when formed in step 2.14 may be or comprise the third emulsion 300 of FIG. 3A and/or the fourth emulsion 302 of FIG. 3B. It is further understood that the second emulsion 204 when formed in step 2.24 may be or comprise the third emulsion 300 of FIG. 3A, the fourth emulsion 302 of FIG. 3B, the fifth emulsion 304 of FIG. 3C, and/or the sixth emulsion 306 of FIG. 3D.

Referring now generally to the Figures and particularly to FIG. 3E, FIG. 3E is a process chart presenting aspects of preparing an ingestible mixture 308 formulated for sublingual or ingested consumption or ingestion by a subject and that includes elements of the method of FIG. 2.

In a first step 2.00 of the method of FIG. 3E, a process of formulating the ingestible mixture 308 is initiated. In step 2.02, the compounder selectively heats one or more of the ingredients disclosed herein to a preferred temperature for the purposes of the effectiveness of the process steps of the method of FIG. 3E. It is understood that the ingredients selected for use in the method of FIG. 3E are preferably maintained within a temperature range between 35 degrees Fahrenheit and 90 degrees Fahrenheit.

In step 3.08 a volume carrier oil 310 is heated and introduced into the process flow the formulation of the ingestible mixture 308, wherein the carrier oil 310 may be or comprise (a.) a volume of Vitis vinifera (Grape) seed oil (a first alternative carrier oil); (b.) a volume of Capric Triglycerides (or, “MCT”) (a second alternative carrier oil); (c.) a volume of Olive oil (at third alternative carrier oil); (d.) a volume of olive oil; and/or (e.) another suitable ingestible oil known in the art.

In step 2.06 the compounder provides and optionally blends together a volume of vitamin E with the volume of carrier oil 310. In optional step 2.14 the compounder emulsifies the volume of vitamin E with the volume of carrier oil 310 to create a seventh emulsion 312. The compounder may optionally use a prior art ultrasonic processor in optional step 2.14 to form the seventh emulsion 312, such as the UIP 1500hdt™ (20 kHz, 15000 W) ultrasonics processor as marketed by Hielscher Ultrasonics, GMBH of Teltow, Germany, or other suitable ultrasonics processor known in the art.

In step 2.16 the compounder further blends a volume of a cannabinoid extract into the seventh emulsion 312. Preferably the volume of cannabinoid extract provided in step 2.16 includes one milligram (hereinafter, “mG”) to 100 mG of cannabinoid and a volume of oil in the range of from 10 milliliter (hereinafter, “mL”) to 30 mL of oil+/−10%.

In optional step 2.20 the compounder further blends a volume of Arnica montana powder or extract into the seventh emulsion 312. In optional step 2.22 the compounder further blends a volume of bromelain into the seventh emulsion 312.

In step 2.24 the compounder further emulsifies the seventh emulsion 312 to create an eighth emulsion 314 from the collection of the ingredients contained and added to the seventh emulsion 312 as of step 2.24. In the emulsification of step 2.24 forming the eighth emulsion 314, the compounder may optionally use the prior art ultrasonic processor of step 2.14, such as the UIP 1500hdt™ (20 kHz, 15000 W) ultrasonics processor as marketed by Hielscher Ultrasonics, GMBH of Teltow, Germany, or other suitable ultrasonics processor known in the art, to form the eighth emulsion 314.

In step 3.10 the compounder elects whether or not to include a volume of Mentha piperita (peppermint oil) to desired variant of the ingestible mixture 308, and proceeds on to step 3.12 when deciding to additionally include a volume of Mentha piperita (peppermint oil) to a desired variant of the ingestible mixture 308.

In the alternative, the compounder proceeds from step 3.10 to step 3.14 when deciding to not include a volume of Mentha piperita (peppermint oil) to the eighth emulsion 312, and thereupon proceeds form step 2.26 to step 2.28.

In step 3.14 the compounder elects whether or not to include a volume of Lavandula angustifolia (lavender flower) oil to a desired variant of the ingestible mixture 308, and proceeds on to step 3.16 when deciding to additionally include a volume of Lavandula angustifolia (lavender flower) oil to a desired variant of the ingestible mixture 308.

In the alternative, the compounder proceeds from step 3.14 or step 3.16 to optional step 2.28. In optional step 2.28 the compounder applies a prior art mixer, such as a DeBEE 3000™ homogenizer mixer marketed by Bee International Corporation of South Easton, Mass., or other suitable mixer or blender known in the art, in an attempt to mix the ingestible mixture 308 to present a color and viscosity. The compounder proceeds from optional step 2.28, optional step 2.30 to step 2.32, whereupon the formulation of the invented oil mixture 22 is designated as completed as one of a wide variety of alternate preferred embodiments of the ingestible mixture 308.

It is understood that the ingestible mixture 308 may contain one or more additional components suitable for ingestion when combined, blended or mixed with the ingredients optionally added in the steps of 3.08 through 3.16.

Referring now generally to the Figures and particularly to FIG. 4, FIG. 4 is an additional process chart of the presenting aspects of preparing alternate preferred embodiments of the invented oil mixture 22 and presenting additional optional and alternative ingredients and process steps of FIG. 2.

In step 4.00, having elected to include one or more optional ingredients, the compounder elects whether or not to include the optional ingredient Lavandula angustifolia (lavender) flower oil into the invented oil mixture 22. In optional step 4.02, the compounder blends a volume of Lavandula angustifolia (lavender) flower oil into the second emulsion 204. In step 4.04 the compounder elects whether or not to include the optional ingredient Mentha piperita (peppermint) oil into the invented oil mixture 22. In optional step 4.06, the compounder blends a volume of Mentha piperita (peppermint) oil into the second emulsion 204. In step 4.08 the compounder elects whether or not to include the optional ingredient of industrial-grade, cosmetic-grade, or pharmaceutical-grade DMSO into the invented oil mixture 22. In optional step 4.10, the compounder blends a volume of cosmetic-grade or pharmaceutical-grade DMSO into the second emulsion 204. In step 4.12 the compounder elects whether or not to include an optional ingredient Populus balsamifera (balsam poplar) into the invented oil mixture 22. In optional step 4.14, the compounder blends a volume of Populus balsamifera (balsam poplar) into the second emulsion 204. In step 4.16 the compounder elects whether or not to include an optional ingredient lidocaine into the invented oil mixture 22. In optional step 4.18, the compounder blends a volume of lidocaine into the second emulsion 204. In step 4.20 the compounder elects whether or not to include a volume of the optional ingredient Hamameus virginiana (witch hazel) into the invented oil mixture 22. In optional step 4.22, the compounder blends the Hamameus virginiana (witch hazel) into the second emulsion 204.

The compounder proceeds from either step 4.20 or optional step 4.22 to step 2.28 of the method of FIG. 2.

Referring now generally to the Figures and particularly to FIG. 5, FIG. 5 is an additional process chart of the presenting aspects of preparing alternate preferred embodiments of the invented oil mixture of FIG. 1 and presenting additional optional process steps of combining the invented oil mixture 22 to a lotion 500, a cream 502, or a gel 504.

In step 5.00, when having elected in step 2.30 to combine the invented oil mixture 22 with a lotion 500, a cream 502, and/or a gel 504, the compounder proceeds to step 5.00 and to determine whether to combine the invented oil mixture 22 with a volume of the lotion 500. When the compounder elects in step 5.00 to combine the invented oil mixture 22 with a volume of lotion 500, the compounder combines the invented oil mixture 22 with the volume of the lotion 500 in optional step 5.02. The compounder proceeds from step 5.02 to step 2.32 of FIG. 2

In an alternative to step 5.00, in step 5.04, the compounder elects whether or not to combine the invented oil mixture 22 with a volume of cream 502. In optional step 5.06, the compounder combines the invented oil mixture 22 with the volume of the cream 502. The compounder proceeds from step 5.06 to step 2.32 of FIG. 2. In an alternative to step 5.04, in step 5.08 the compounder elects whether or not to combine the invented oil mixture 22 with a volume of gel 504. When the compounder elects in step 5.08 to combine the invented oil mixture 22 with a volume of gel 504, the compounder combines the invented oil mixture 22 with the volume of the gel 504 in optional step 5.10. The compounder proceeds from either step 5.08 or step 5.10 to step 2.32 of FIG. 2.

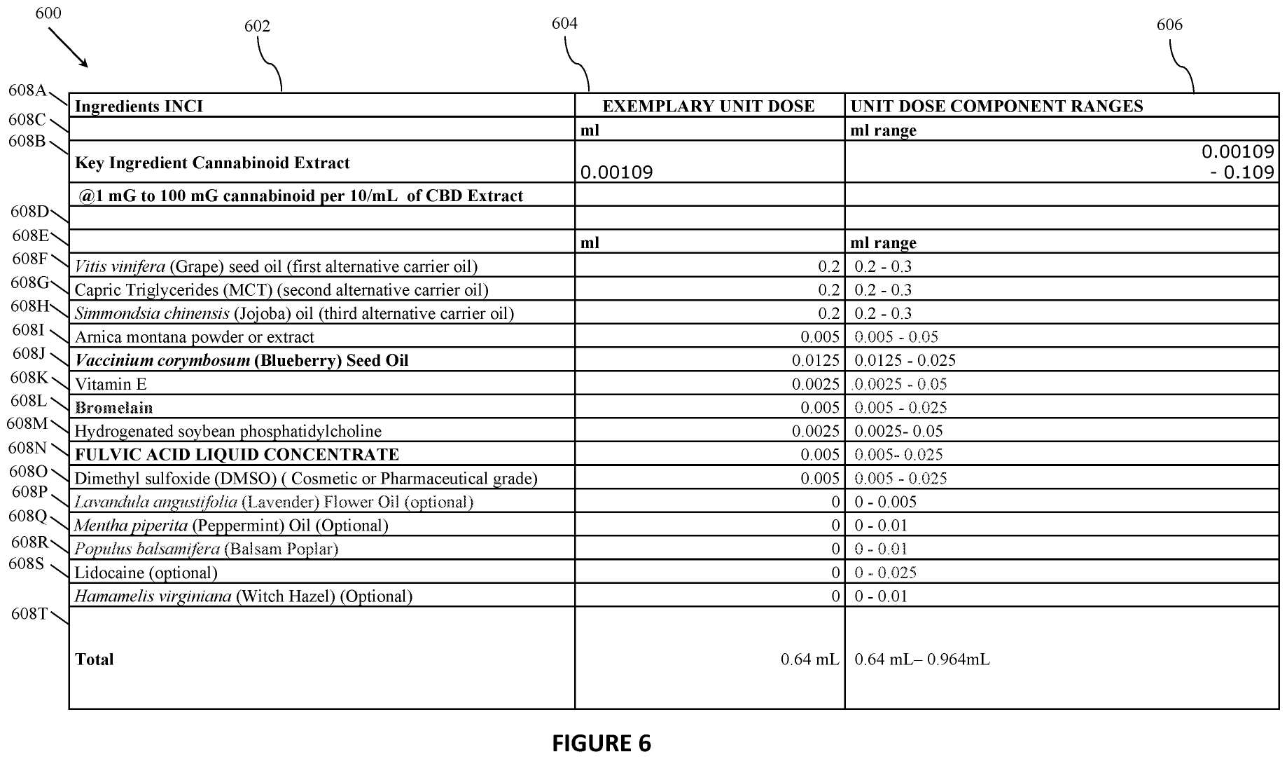

Referring now generally to the Figures and particularly to FIG. 6, FIG. 6 is a formulary table 600 of unit dose formulation listings and presenting both an exemplary unit dose formulation of alternate preferred embodiments of the invented oil mixture 22. Various possible, alternative and optional ingredients of alternate preferred embodiments of the invented oil mixture 22 are listed in a first column 602. Exemplary ingredient measures are listed in a second column 604 and are associated by inclusion in a same row with individual alternative and optional ingredients. Ranges of ingredient magnitudes of said alternative and optional ingredients thereof in presented a third column 606 and are associated by inclusion in a same row with individual alternative and optional ingredients. A header row 608A labels the first column 602, the second column 604, and the third column 606 for easy legibility of the table 600.

A row 608B indicates that, for the Key Ingredient cannabinoid extract, a first exemplary unit dose is 0.00109 milliliter and a preferred unit dose range is 0.00109 milliliter-0.109 milliliter and includes a cannabinoid component preferably in the range of from 1 mG to 100 mG. A row 608C directly above the row 608B labels units for the values in the row 608B. A row 608D indicates that the measurements for Exemplary Unit Dose given below this row in the column 604 are given in milliliters, and the measurements for Unit Dose Component Ranges given below this row in the column 606 are each a range that is measured in milliliters. A row 608E lists that the exemplary unit dose for the ingredient Vitis vinifera (Grape) Seed Oil is 0.2 mL and the unit dose component range is 0.2 mL-0.3 mL. A row 608F lists that the exemplary unit dose for the ingredient Capric Triglycerides (MCT) (a second alternative carrier oil) is 0.2 mL and the unit dose component range is 0.2 mL-0.3 mL. A row 608G lists that the exemplary unit dose for the ingredient Simmondsia chinensis (Jojoba) oil (a third alternative carrier oil) is 0.2 mL and the unit dose component range is 0.2 mL-0.3 mL. A row 608H lists that the exemplary unit dose for the ingredient Arnica montana powder or extract is 0.005 mL and the unit dose component range is 0.005 mL-0.05 mL. A row 608I lists that the exemplary unit dose for the ingredient Vaccinium corymbosum (Blueberry) Seed Oil is 0.0125 mL and the unit dose component range is 0.0124 mL-0.025 mL. A row 608J lists that the exemplary unit dose for the ingredient Vitamin E is 0.0025 mL and the unit dose component range is 0.0025-0.05 mL. A row 608K lists that the exemplary unit dose for the ingredient Bromelain is 0.005 mL and the unit dose component range is 0.005 mL-0.025 mL. A row 608L lists that the exemplary unit dose for the ingredient Hydrogenated soybean phosphatidylcholine is 0.0025 mL and the unit dose component range is 0.0.0025 mL-0.05 mL. A row 608M lists that the exemplary unit dose for the ingredient Fulvic Acid Liquid Concentrate is 0.005 mL and the unit dose component range is 0.005 mL-0.025 mL. A row 608N lists that the exemplary unit dose for the ingredient DMSO (of industrial, cosmetic or pharmaceutical grade) is 0.005 mL and the unit dose component range is 0.0.005 mL-0.025 mL. A row 608O lists that the exemplary unit dose for the ingredient Lavandula angustifolia (Lavender) Flower Oil may be zero and the unit dose component range is up to 0.005 mL. A row 608P lists that the exemplary unit dose for the ingredient Mentha piperita (Peppermint) Oil may be zero and the unit dose component range is up to 0.01 mL. A row 608Q lists that the exemplary unit dose for the ingredient Populus balsamifera (Balsam Poplar) may be zero and the unit dose component range is up to −0.01 mL. A row 608R lists that the exemplary unit dose for the ingredient Lidocaine may be zero and the unit dose component range is. is up to 0.025 mL. A row 608S lists that the exemplary unit dose for the ingredient Hamamelis virginiana (Witch Hazel) may be zero and the unit dose component range is up to 0.01 mL. A row 608T lists that the total volume of the listed ingredients in the exemplary unit dose combined may be 0.2373 mL, and the total volume of the combined ingredients will range 0.64 mL-0.964 mL.

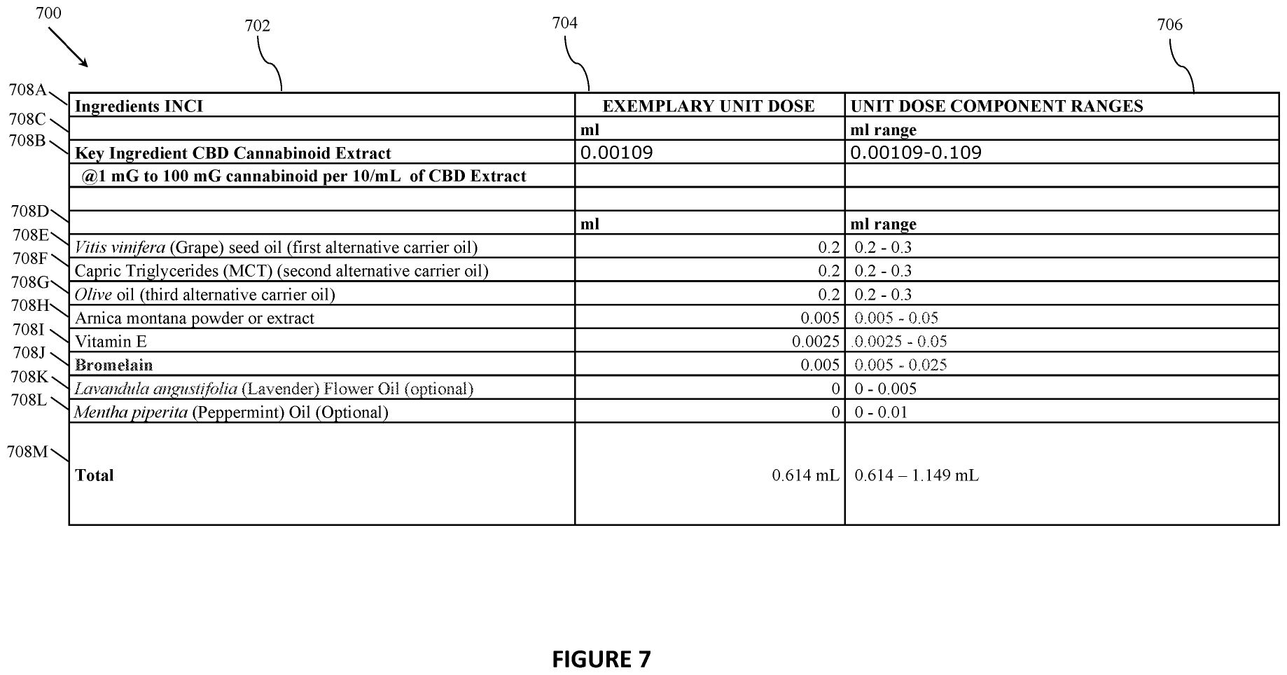

Referring now generally to the Figures and particularly to FIG. 7, FIG. 7 is a ingestible formulary table 700 of unit dose formulation listings and presenting both an exemplary unit dose formulation of alternate preferred embodiments of the invented ingestible mixture 308. Various possible, alternative and optional ingredients of alternate preferred embodiments of the invented ingestible mixture 308 are listed in a first column 702. Exemplary ingredient measures are listed in a second column 704 and are associated by inclusion in a same row with individual alternative and optional ingredients. Ranges of ingredient magnitudes of said alternative and optional ingredients thereof in presented a third column 706 and are associated by inclusion in a same row with individual alternative and optional ingredients. A header row 708A labels the first column 702, the second column 704, and the third column 706 for easy legibility of the table 700. A row 708B indicates that, for the key ingredient cannabinoid extract, a first exemplary unit dose is 0.00109 milliliter and the unit dose range is 0.00109 milliliter-0.109 milliliter and includes a cannabinoid component preferably in the range of from mG to 100 mG.

A row 708C directly above the row 708B labels units for the values in the row 708B. A row 708D indicates that the measurements for an exemplary ingestible unit dose given below this row in the column 704 are given in milliliters, and the measurements for ingestible unit dose component ranges given below this row in the column 706 are each expressed measured in milliliters.

A row 708E lists that the exemplary unit dose for the ingredient Vitis vinifera (Grape) Seed Oil is 0.2 mL and the unit dose component range is 0.2 mL-0.3 mL. A row 708F lists that the exemplary unit dose for the ingredient Capric Triglycerides (MCT) (second alternative carrier oil) is 0.2 mL and the unit dose component range is 0.2 mL-0.3 mL. A row 708G lists that the exemplary unit dose for the ingredient Olive oil (third alternative carrier oil) is 0.2 mL and the unit dose component range is 0.2 mL-0.3 mL. A row 708H lists that the exemplary unit dose for the ingredient Arnica montana powder or extract is 0.005 mL and the unit dose component range is 0.005 mL-0.05 mL. A row 708I lists that the exemplary unit dose for the ingredient Vitamin E is 0.0025 mL and the unit dose component range is 0.0025-0.05 mL. A row 708J lists that the exemplary unit dose for the ingredient Bromelain is 0.005 mL and the unit dose component range is 0.005 mL-0.025 mL. A row 708K lists that the exemplary unit dose for the ingredient Lavandula angustifolia (Lavender) Flower Oil may be zero and the unit dose component range is up to 0.005 mL. A row 708L lists that the exemplary unit dose for the ingredient Mentha piperita (Peppermint) Oil may be zero and the unit dose component range is up to 0.01 mL. A row 708M lists that the total volume of the listed ingredients in an exemplary ingestible unit dose combined may be 0.614 mL, and the total volume of the combined ingredients will range 0.614 mL-1.149 mL.

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part”, “section”, “portion”, “member”, or “element” when used in the singular can have the dual meaning of a single part or a plurality of parts. Finally, terms of degree such as “substantially”, “about”, and “approximately” as used herein mean a reasonable amount of deviation of the modified term such that the end result is not significantly changed.

While selected embodiments have been chosen to illustrate the invented system, it will be apparent to those skilled in the art from this disclosure that various changes and modifications can be made herein without departing from the scope of the invention as defined in the appended claims. For example, the size, shape, location, or orientation of the various components can be changed as needed and/or desired. Components that are shown directly connected or contacting each other can have intermediate structures disposed between them. The functions of one element can be performed by two, and vice versa. The structures and functions of one embodiment can be adopted in another embodiment, it is not necessary for all advantages to be present in a particular embodiment at the same time. Every feature which is unique from the prior art, alone or in combination with other features, also should be considered a separate description of further inventions by the applicant, including the structural and/or functional concepts embodied by such feature(s). Thus, the foregoing descriptions of the embodiments according to the present invention are provided for illustration only, and not for the purpose of limiting the invention as defined by the appended claims and their equivalents.

1. A transdermal pharmaceutical composition for delivery of cannabinoid into the systemic circulation comprising:
(a) a mixture of Vaccinium corymbosum seed oil, bromelain, and fulvic acid;
(b) a volume of soybean oil derivative; and
(c) a volume of cannabinoid.

2. The transdermal pharmaceutical composition of claim 1, wherein the cannabinoid comprises cannabidiol.

3. The transdermal pharmaceutical composition of claim 1, wherein the cannabinoid is present in a range of from 1 mg to 100 mg of the pharmaceutical composition.

4. The transdermal pharmaceutical composition of claim 1, further comprising a nanoliposomal delivery mixture.

5. The transdermal pharmaceutical composition of claim 1, further comprising a volume of Arnica montana powder or extract.

6. The transdermal pharmaceutical composition of claim 1, further comprising Vaccinium corymbosum seed oil.

7. The transdermal pharmaceutical composition of claim 1, further comprising a volume of Vitamin E.

8. The transdermal pharmaceutical composition of claim 1, further comprising a volume of Lavandula angustifolia oil.

9. The transdermal pharmaceutical composition of claim 1, further comprising a volume of Mentha piperita.

10. The transdermal pharmaceutical composition of claim 1, further comprising a volume of Populus balsamifera.

11. The transdermal pharmaceutical composition of claim 1, further comprising a volume of lidocaine.

12. The transdermal pharmaceutical composition of claim 1, further comprising a volume of Hamamelis virginiana.

13. The transdermal pharmaceutical composition of claim 1, further comprising an additional permeation enhancer selected from the permeation enhancer group consisting of DMSO and Vitis vinifera seed oil.

14. The transdermal pharmaceutical composition of claim 1, wherein the pharmaceutical composition further comprises one or more pharmaceutically acceptable excipients to create a transdermal dosage form selected from the group consisting of: gels, ointments, cataplasms, poultices, pastes, creams, lotions, plasters, and jellies.

15. (canceled)

16. (canceled)

17. (canceled)

18. A transdermal pharmaceutical composition for delivery of cannabinoid into the systemic circulation comprising:
(a) a mixture of Vaccinium corymbosum seed oil, bromelain, and fulvic acid;
(b) a volume of cannabinoid; and
(c) a volume of a nanoliposomal delivery substance.

19. A transdermal pharmaceutical composition for delivery of cannabinoid into the systemic circulation comprising:
(a) a mixture of Vaccinium corymbosum seed oil, bromelain, and fulvic acid;
(b) a volume of cannabinoid; and
(c) a volume of a DMSO.

20. (canceled)

21. (canceled)

22. (canceled)

23. (canceled)

24. A transdermal pharmaceutical composition for delivery of cannabinoid into the systemic circulation comprising:
a volume of Vaccinium corymbosum seed oil;
a volume of a cannabinoid extract selected from the extract group consisting of CBD and THC;
a volume of Bromelain;
a volume of Arnica; and
a volume of fulvic acid.

25. The transdermal pharmaceutical composition for delivery of cannabinoid into the systemic circulation of claim 24, further comprising:
a volume of carrier oil selected form the carrier oil group consisting of capric triglycerides, Vitis vinifera seed oil, Simmondsia chinensis oil, and olive oil;
a volume of vitamin E; and
a flavoring volume selected from the flavor group of Lavender flower oil and peppermint oil.
